(19) United States
(12) Patent Application Publication
(10) Pub. No.: US 2026/0191926 A1
(43) Pub. Date: Jul. 9, 2026

(54) MC4 Antagonists in the Treatment of Restless Legs Syndrome

(71) Applicant: YALE UNIVERSITY, New Haven, CT (US)

(72) Inventor: Brian Koo, New Haven, CT (US)

(21) Appl. No.: 19/444,512

(22) Filed: Jan. 9, 2026

Related U.S. Application Data
(60) Provisional application No. 63/743,418, filed on Jan. 9, 2025.

(51) Int. Cl. A61K 38/12; A61K 31/4164; A61K 31/4535; A61K 31/4545; A61K 31/4725; A61K 31/496; A61K 31/506; A61K 45/06; A61P 25/14

(52) U.S. Cl. CPC A61K 38/12; A61K 31/4164; A61K 31/4535; A61K 31/4545; A61K 31/4725; A61K 31/496; A61K 31/506; A61K 45/06; A61P 25/14

(57) ABSTRACT

Provided herein are methods of treating restless leg syndrome (RLS). The methods include administering to a subject a pharmaceutical composition containing an MC4 antagonist or a pharmaceutically acceptable salt thereof.

### CROSS-REFERENCE TO RELATED APPLICATIONS

This application claims priority to U.S. Provisional Patent Application No. 63/743,418 entitled “MC4 ANTAGONISTS IN THE TREATMENT OF RESTLESS LEGS SYNDROME,” filed Jan. 9, 2025, the disclosure of which is incorporated herein by reference in its entirety.

### STATEMENT REGARDING FEDERALLY SPONSORED RESEARCH

This invention was made with government support under number W81XWH2010003 awarded by the Department of Defense. The government has certain rights in the invention.

### SEQUENCE LISTING

This application contains one or more sequences in a computer readable format in an accompanying XML file. The Sequence Listing submitted herewith is contained in an XML file named “047162-7601US1_Seq.xml,” created on Jan. 8, 2026 and having a size of 6,077 bytes, which is incorporated herein by reference in its entirety.

### BACKGROUND

Currently, restless leg syndrome (RLS) affects approximately 5 million adults in the U.S. RLS is a common sleep disorder characterized by an urge to move that occurs at night and prevents sleep. The cause of RLS is not known. The most commonly prescribed medicines, dopamine agonists, cause a paradoxical worsening of RLS over years, called RLS augmentation. To treat patients with augmentation, the dopamine agonists must be tapered, but this comes with great distress and a prolonged but temporary RLS worsening. Drugs are available to subsequently treat RLS, but these include largely opioid medications.

Furthermore, there is no treatment to ease the universal suffering patients must face when tapering off of dopamine agonists. A treatment to ameliorate RLS during augmentation, particularly when weaning from dopamine agonist is critically needed. The present disclosure solves this unmet need.

### BRIEF SUMMARY OF THE INVENTION

Provided herein is a method of treating, preventing, and/or ameliorating restless leg syndrome (RLS) in a subject in need thereof. Also provided, in various aspects, are methods of treating, preventing, and/or ameliorating augmentation associated with prior treatment of RLS in a subject in need thereof. Also provided, in various aspects, are methods of reducing and/or eliminating symptoms associated with RLS in a subject in need thereof.

In certain embodiments, the method comprises administering to the subject a therapeutically effective amount of an MC4 antagonist or a pharmaceutically acceptable salt thereof. In certain embodiments, the MC4 antagonist is formulated as a pharmaceutical composition further comprising at least one pharmaceutically acceptable carrier or excipient. In various aspects, the MC4 antagonist is a small-molecule, peptide, or antibody.

In certain aspects, the subject of the methods described herein is identified by measuring in the subject a (α-MSH+β-MSH):β-endorphin ratio of at least about 5 prior to administering the MC4 antagonist to the subject.

In certain aspects, the subject of the methods described herein is identified by measuring in the subject a β-MSH:β-endorphin ratio of at least about 3 prior to administering the MC4 antagonist to the subject.

### BRIEF DESCRIPTION OF THE FIGURES

FIGS. 1A-1C show nighttime plasma melanocortin levels in restless legs syndrome (RLS) and control cohorts, which are shown as mean and standard deviation in picograms per milliliter of (FIG. 1A) adrenocorticotropin hormone (ACTH) in RLS (n=40, 40, 39) compared to controls (n=42, 42, 41), (FIG. 1B) α-melanocyte-stimulating hormone (α-MSH) in RLS (n=41, 38, 38) compared to controls (n=40, 40, 39), and (FIG. 1C) β-endorphin (β-EDP) in RLS (n=38, 41, 40) compared to controls (n=43, 44, 43) at 17:00, 20:30, and 22:00 in RLS (black circles) and controls (open squares; n=N_{17:00}, N_{20:30}, N_{22:00}). There were no differences between RLS and control groups at any time point for plasma ACTH, α-MSH, or β-EDP. Error bars indicate standard deviation.

FIGS. 2A-2B show plasma prohormone pro-opiomelanocortin (POMC) levels in femtomoles per liter at 22:00 for (FIG. 2A) restless legs syndrome (RLS; black; n=39) and controls (white; n=39) and (FIG. 2B) nonpainful RLS (light gray; n=30), painful RLS (dark gray; n=9), and controls (white; n=39). FIG. 2A shows that plasma POMC levels are significantly higher in RLS patients compared to controls (17.0±11.5 vs 12.7±6.1, p=0.048). FIG. 2B shows that plasma POMC levels are significantly different among RLS patients with nonpainful RLS, RLS patients with painful RLS, and controls (18.7±12.1 vs 11.2±7.4 vs 12.7±6.1, analysis of variance p=0.01). Plasma POMC levels are significantly higher in RLS patients with nonpainful RLS compared to RLS patients with painful RLS (18.7±12.1 vs 11.2±7.4, p=0.04) and also compared to controls (18.7±12.1 vs 12.7±6.1, p=0.02). *p<0.05; error bars indicate standard deviation.

FIGS. 3A-3C show cerebrospinal fluid (CSF) α-melanocyte-stimulating hormone (α-MSH), β-MSH, and β-endorphin (β-EDP) levels, which are shown in picograms per milliliter, in restless legs syndrome (RLS; black), painful RLS (dark gray), nonpainful RLS (light gray), and controls (white). (FIG. 3A) CSF α-MSH was higher in RLS patients than controls (34.2±40.9 vs 20.3±11.0, p=0.062). CSF α-MSH was highest in nonpainful RLS (n=26), intermediate in painful RLS (n=8), and lowest in controls (n=34), although this was not statistically significant (36.0±45.3 vs 28.5±23.0 vs 20.3±11.0, analysis of variance [ANOVA] p=0.14). (FIG. 3B) CSF β-MSH was similar between RLS and controls (36.2±18.9 vs 32.6±15.2, p=0.38). CSF β-MSH was higher in painful RLS (n=9) than in nonpainful RLS (n=26) or in controls (n=35; 48.2±24.8 vs 32.1±14.8 vs 32.6±15.2, ANOVA p=0.03). (FIG. 3C) CSF β-EDP was lower in the RLS compared to the control cohort (10.3±3.2 vs 12.3±5.0, p=0.059). CSF β-EDP was lowest in painful RLS (n=7), intermediate in nonpainful RLS (n=27), and highest in controls (n=34; 8.0±3.4 vs 10.8±3.1 vs 12.3±5.0, ANOVA p=0.049). With post hoc testing, those with painful RLS had significantly lower CSF β-EDP than controls (8.0±3.4 vs 12.3±5.0, p=0.02). *p<0.05 between 2 groups using Student t test; **over the <-> refers to p<0.05 among 3 groups using ANOVA test. Error bars indicate standard deviation.

FIG. 4 is a schematic showing the processing of the prohormone pro-opiomelanocortin (POMC). The prohormone POMC undergoes extensive post-translational processing and is cleaved to yield γ-melanocyte-stimulating hormone (γ-MSH), adrenocorticotropin hormone (ACTH), and β-lipotropin. ACTH is cleaved to give α-MSH and corticotropin-like intermediate peptide (CLIP), whereas β-lipotropin is broken down into γ-lipotropin and β-endorphin. Finally, γ-lipotropin is cleaved into β-MSH.

### DETAILED DESCRIPTION OF THE INVENTION

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

Restless legs syndrome (RLS) is a common sensorimotor sleep disorder characterized by an unrelenting urge to move, occurring at night during periods of inactivity, when patients assume recumbency for sleep. RLS and its canonical urge to move prevent sleep by forcing the sufferer to move or walk during times of rest when sleep is most desired. RLS is the most common neurologically based sleep disorder with a prevalence of 7.2% in North American and European populations. The disease is chronic and progressive and in moderate-to-severe RLS, when symptoms occur at least twice weekly, lifelong medical therapy is needed. Despite this, treatment options for RLS are finite and the medicines most commonly used to treat RLS actually cause a paradoxical worsening of RLS over months to years in the clinical phenomenon of augmentation. The use of these medicines, the dopamine agonists, in part was popularized by an erroneous pathobiologic assumption of dopamine deficiency in RLS, whereas it is now known that dopamine exists in excess in the brain of RLS patients. This clinical entity of augmentation exposes a pathobiology-treatment mismatch and underscores the critical need to further clarify the pathobiology of RLS.

RLS is characterized by an urge to move and sensory discomfort that arise at night, suggesting that a biological substance with diurnal release may underlie RLS. Hormones of the melanocortin (MC) system are produced in the hypothalamic arcuate nucleus, are released diurnally, and possess properties that parallel these core features of RLS. The MC system prohormone, pro-opiomelanocortin (POMC), is cleaved to yield adrenocorticotropin hormone (ACTH) and β-lipotropin, which subsequently are cleaved to yield the neuropeptides α-melanocyte-stimulating hormone (α-MSH) and β-endorphin (β-EDP), respectively. The main MC agonist, α-MSH, stimulates excessive locomotion and grooming in rodents as well as a state of hyperalgesia, when administered centrally. Alternately, the actions of β-EDP oppose those of α-MSH, as it promotes passivity and analgesia. Like α-MSH, ACTH administered into periaqueductal gray matter of rats provokes motor behavior and hyperalgesia. In various embodiments, the rodent RLS model through intracerebroventricular administration of α-MSH or ACTH, which caused increased locomotion and grooming, fragmented sleep, and periodic hindlimb movements during sleep, recapitulating core RLS features.

### MC4 Antagonist Compounds

MC4 antagonist compounds described herein can be prepared using the synthetic method known by those skilled in the art. The MC4 antagonist can be a small molecule, a peptide, or an antibody. In various embodiments, the small molecule is a non-peptidic small molecule.

Non-limiting examples of suitable MC4-antagonists include TCMCB07, PF-07258669, ML 00253764, and HS024, the structures of which are shown below.

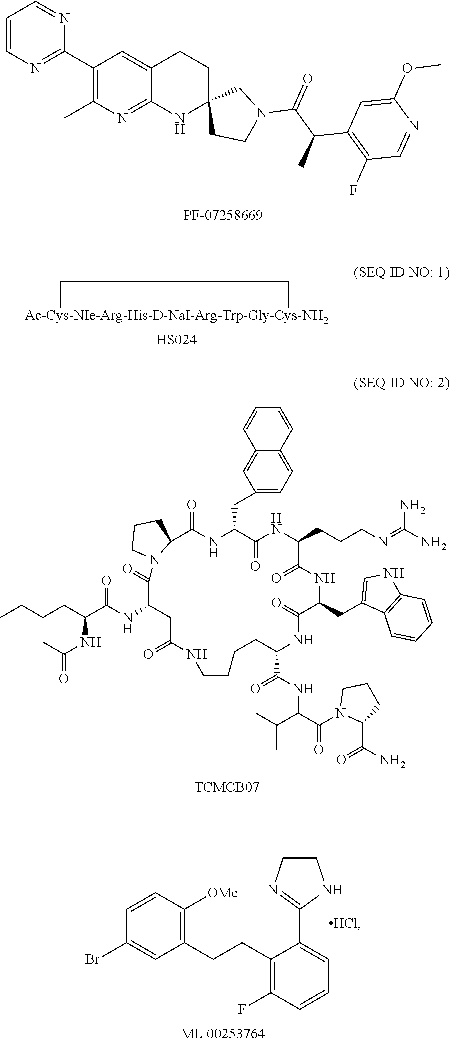

or a pharmaceutically acceptable salt thereof.

Other small-molecule MC4 antagonists include, but not limited to:

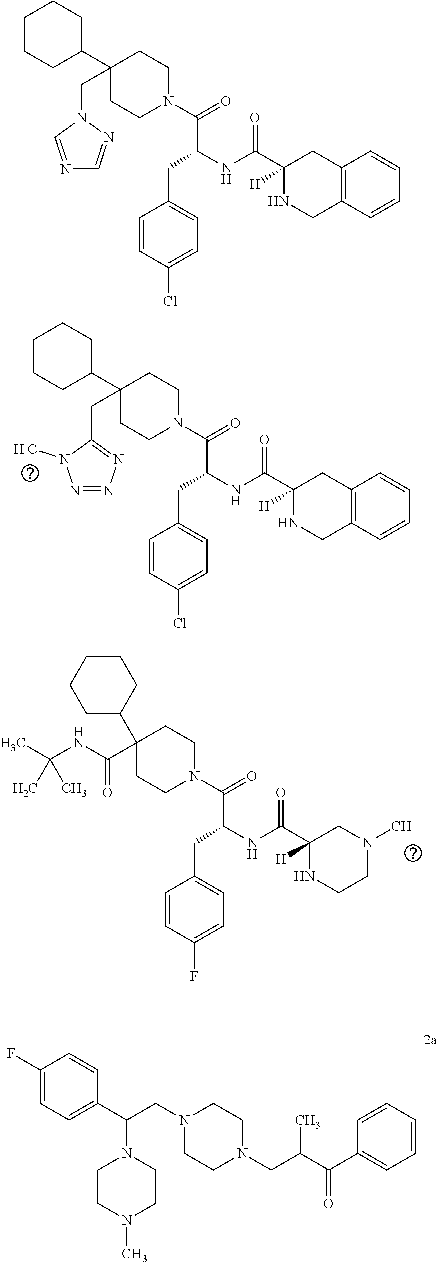

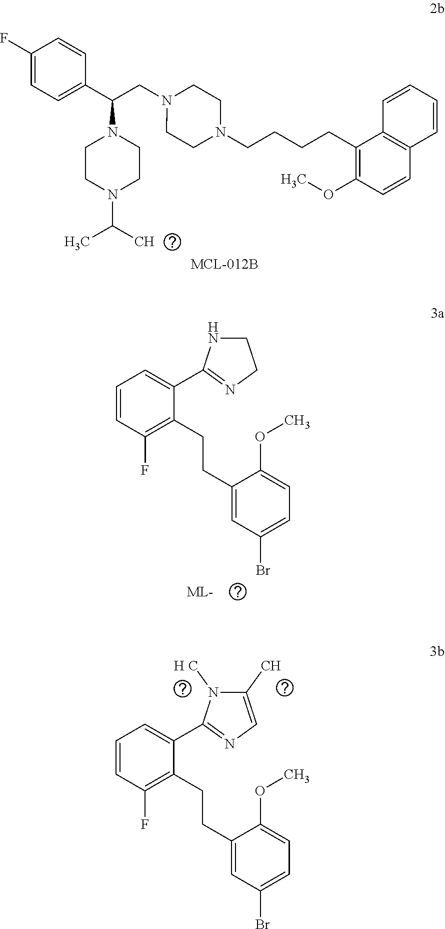

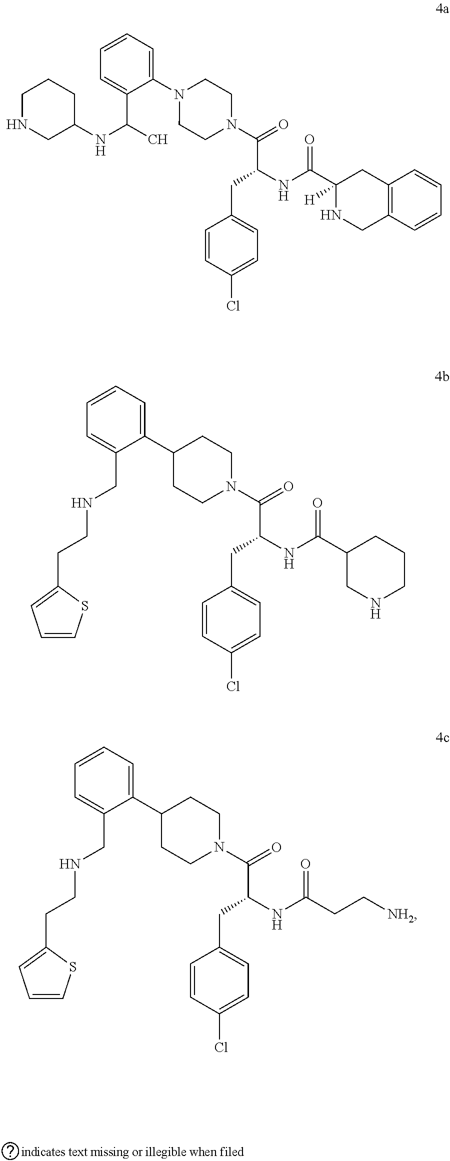 [SMILES: CC(NC1CCCNC1)c1ccccc1N1CCN(C(=O)[C@@H](Cc2ccc(Cl)cc2)NC(=O)[C@H]2Cc3ccccc3CN2)CC1 ; O=C(N[C@H](Cc1ccc(Cl)cc1)C(=O)N1CCC(c2ccccc2CNCCc2cccs2)CC1)C1CCCNC1 ; NCCC(=O)N[C@H](Cc1ccc(Cl)cc1)C(=O)N1CCC(c2ccccc2CNCCc2cccs2)CC1]

or a pharmaceutically acceptable salt thereof.

In various embodiments, the MC4 antagonist is the only pharmaceutically active agent in the pharmaceutical composition. In various embodiments, the pharmaceutical composition used in the methods described herein excludes, and is free of, fatty acids such as C₁₀-C₂₀ fatty acids, as well as esters or other prodrugs thereof.

As used herein, the term “non-peptidic” means that the moiety in question does not contain any naturally occurring amino acids. The non-peptidic small molecule, in some embodiments, has a molecular mass (independent of any connecting fragments or linkers that may be present) of at least 50, 60, 70, 80, 90, 100, 110, 120, or 130 g/mol. The non-peptidic small molecule, in some embodiments, has a molecular mass (independent of any connecting fragments or linkers that may be present) of not greater than 500, 520, 540, 560, 580, 600, 620, 640, 660, 680, 700, 720, 740, 760, 780, 800, 820, 840, 860, 880, or 900 g/mol.

The MC4 antagonist non-peptidic small molecule, in various embodiments, has an in vitro or in vivo potency (as measured by IC₅₀ or EC₅₀) against its target receptor of less than, at least, or equal to about 20, 15, 10, 9, 8, 7, 6, 5, 4, 3, 2 or about 1 μM. The MC4 antagonist non-peptidic small molecule, in various embodiments, has an in vitro or in vivo potency (as measured by IC₅₀ or EC₅₀) against its target receptor of less than, at least, or equal to about 900, 800, 700, 600, 500, 400, 300, 200, 100, 90, 80, 70, 60, 50, 40, 30 20, 10, 9, 8, 7, 6, 5, 4, 3, 2, 1, 0.5, 0.1, 0.05, or about 0.01 nM.

In various embodiments, an MC4 antagonist peptide contains 4 to 50 amino acids, or 4 to 40 amino acids, or 4 to 30 amino acids, 4 to 20 amino acids, or 4 to 10 amino acids. The MC4 antagonist peptide, in various embodiments, has an in vitro or in vivo potency (as measured by IC₅₀ or EC₅₀) against its target receptor of less than, at least, or equal to about 20, 15, 10, 9, 8, 7, 6, 5, 4, 3, 2 or about 1 μM. The MC4 antagonist peptide, in various embodiments, has an in vitro or in vivo potency (as measured by IC₅₀ or EC₅₀) against its target receptor of less than, at least, or equal to about 900, 800, 700, 600, 500, 400, 300, 200, 100, 90, 80, 70, 60, 50, 40, 30 20, 10, 9, 8, 7, 6, 5, 4, 3, 2, 1, 0.5, 0.1, 0.05, or about 0.01 nM. Non-limiting examples of suitable MC4 antagonist peptides include HS014, HS024, JKC363, MBP10, TCMCB07, PG-934, and SBL-MC-31.

If an MC4 antibody is used, then the MC4 antibody has a has an in vitro or in vivo potency (as measured by IC₅₀ or EC₅₀) against its target receptor of less than, at least, or equal to about 900, 800, 700, 600, 500, 400, 300, 200, 100, 90, 80, 70, 60, 50, 40, 30 20, 10, 9, 8, 7, 6, 5, 4, 3, 2, 1, 0.5, 0.1, 0.05, or about 0.01 nM.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

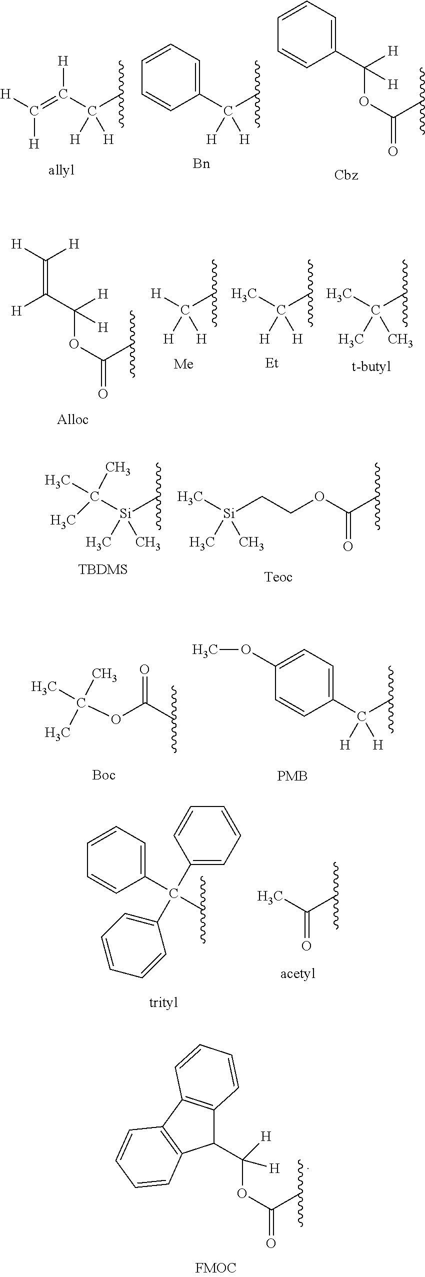

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In various embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Methods of Treating or Ameliorating Restless Leg Syndrome (RLS) and Augmentation Associated with RLS

POMC-derived (pro-opiomelanocortin) peptides were measured in blood and CSF to determine whether the MSH peptides are increased and β-EDP decreased in RLS patients compared to age-/sex-matched controls. The major findings demonstrated higher CSF α-MSH and lower CSF β-EDP in RLS patients, although results did not differ with statistical significance. Following RLS subtyping, those with painful RLS had the lowest, nonpainful RLS intermediate, and controls the highest CSF β-EDP levels. CSF β-MSH was significantly higher in those with painful RLS than in those with nonpainful RLS or controls. There were statistically significant differences in the ratios of CSF β-MSH to CSF β-EDP and of CSF α-MSH+β-MSH to CSF β-EDP among groups, with ratios being highest in painful RLS. In plasma, ACTH, α-MSH, and β-EDP were similar between groups at all time points. Plasma POMC at 22:00 was significantly higher in RLS than controls. Considering these data as a whole, nonpainful RLS is associated with increased plasma POMC and increased CSF α-MSH, perhaps reflecting increased MC tone. Painful RLS is associated with normal plasma POMC, yet increased CSF β-MSH and decreased CSF β-EDP, not reflecting increased MC tone, yet a shift toward β-MSH and away from β-EDP, which both arise from β-lipotropin cleavage.

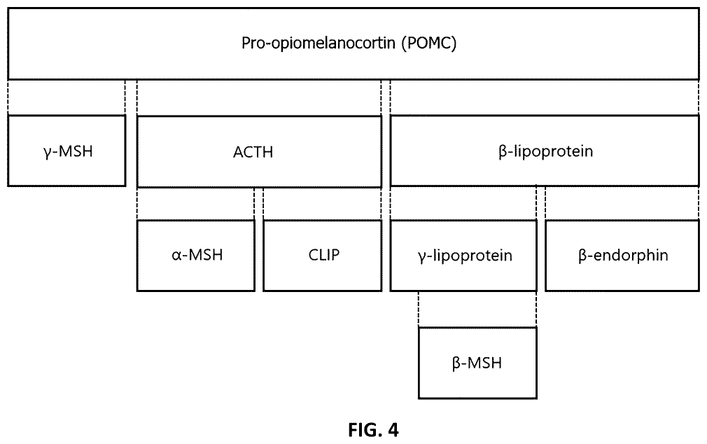

This disclosure provides evidence that MC hormone levels are altered in subjects suffering from RLS. POMC undergoes various cleavage steps to yield the peptides γ-MSH, ACTH, and β-lipotropin. ACTH is further cleaved to α-MSH, whereas β-lipotropin is cleaved to β-MSH and β-EDP (FIG. 4). POMC is highly conserved across higher vertebrate species and underlies basic life behaviors/functions, which include feeding, sex, movement, and pain sensitivity. Study of MC-related behavioral effects date back to 1955 and descriptions of the ACTH-induced stretching and yawning syndrome. In this syndrome, animals appear tired, while stretching and yawning repeatedly, reminiscent of the tiredness and desire to stretch that RLS sufferers report. Yawning and stretch are also induced by intracerebral α-MSH or β-MSH. The most extensively described of the MC-related behaviors is excessive grooming and locomotion, caused by central α-MSH, β-MSH, or ACTH administration. Excessive locomotion is the animal corollary of the urge to move, which compels the RLS sufferer to walk at night, giving them the colloquial title, “nightwalker.” Sleep following this initial state of α-MSH-induced hyperactivity displays periodic cortical arousal and periodic hindlimb movements, similar to the RLS-related phenomena of periodic limb movements during sleep and arousal. Centrally administered ACTH and α-MSH result in a decreased time to tail-flick in response to a painful thermal stimulus, reflecting hyperalgesia. RLS is experienced as painful in approximately 20% of cases and dysesthetic in the majority of cases. Moreover, persons with RLS exhibit hyperalgesia to pinprick.

Although the MSH peptides and β-EDP arise from the same prohormone, POMC, their actions are opposing. The MSH peptides promote movement and pain hypersensitivity, whereas β-EDP decreases locomotion and pain. Intracerebroventricular β-EDP and nucleus solitarius microinjection enhance slow wave sleep and produce analgesia, inhibiting ACTH-/α-MSH-induced hyperalgesia. Thus, CSF β-EDP being low in RLS and lowest in painful RLS is fitting, as are findings of high CSF α-MSH in RLS given the promotor properties of MC agonists. In nonpainful RLS, both plasma POMC and CSF α-MSH were increased, which may reflect generally increased MC activity. In painful RLS, neither plasma POMC nor CSF α-MSH were increased; rather, CSF β-MSH was increased and CSF β-EDP was decreased. Both β-MSH and β-EDP are β-lipotropin cleavage products, so the abnormality in painful RLS may be a shift toward β-MSH at the expense of β-EDP, not an overall increase in MC activity.

Although the MSH peptides and β-EDP originate from the same transcript, POMC undergoes extensive post-translational processing in the hypothalamic arcuate nucleus and pituitary. At the cellular level, POMC is ferried to the endoplasmic reticulum, where it is amidated and marked for transport to the trans-Golgi network. Here, vesicles bud to produce secretory granules, where proteolytic processing occurs by diverse regulatory mechanisms, yielding varying degrees of ACTH and β-lipotropin, then secretion of variable amounts of MSH peptide and β-EDP. In this way, both increased and decreased levels of different POMC-derived peptides can occur in the same individual and in RLS a favoring toward MC and away from β-EDP activity. The data described herein reflect this, as ratios of α-MSH+β-MSH to β-EDP are increased in RLS.

The ratio of CSF α-MSH+β-MSH to β-EDP was highest in painful RLS, intermediate in nonpainful RLS, and lowest in controls. Without being bound by theory, this result was driven by at least 3 factors. First, CSF β-EDP was lowest, intermediate, and highest in painful, nonpainful RLS, and controls, respectively; second, CSF α-MSH was higher in RLS than controls; and third, CSF β-MSH was higher in painful than nonpainful RLS or controls. Whereas CSF α-MSH was similar in RLS subtypes, CSF β-MSH was higher in painful than nonpainful RLS. Biochemically, both α-MSH and β-MSH show affinity for the MC-1 and MC-4 receptors, the activity of which underlies hyperalgesia and non-neuropathic and neuropathic pain, respectively. We focused on α-MSH and β-MSH, but not 7-MSH, as the latter MSH peptide has little pain-modulating activity. Given their pain-modulating involvement, α- and β-MSH may act synergistically, with pain sensitivity being greatest when both MSH peptides are elevated, in particular when β-EDP is low. As reflected in an increased ratio of CSF α-MSH+β-MSH to β-EDP, an increase in α-MSH and β-MSH and decreased β-EDP would favor motor activity and hyperalgesia over passivity and analgesia.

RLS pharmacology is also consistent with MC-endorphin RLS pathobiology. RLS treatment with L-dopa was described in 1982. Dopamine inhibits ACTH and α-MSH secretion from the pituitary, and inhibits α-MSH acetylation to active α-MSH, thereby decreasing MSH tone and treating RLS. Opioid medications are used mostly in medication-refractory RLS. Like β-EDP, opioids bind μ-opioid receptors to produce analgesia and passivity. In the present study, CSF β-EDP was lowest in painful RLS. Given that β-EDP deficiency may be a cause of RLS, it is understandable why opioid medications treat RLS.

In various embodiments, only subjects/patients with moderate-to-severe RLS were included to increase chances of seeing hormonal abnormalities. Patients taking RLS medicines, specifically dopamine agonists, alpha-2 delta ligands, or opioids, were ineligible or had to stop medications for 7 days, as these medicines could influence MC hormone levels. It should be noted that only 2 RLS patients had to be tapered from an opioid medication, one from the painful RLS group and the other from the nonpainful RLS group. The CSF sample size of 37 RLS and 36 control subjects was large and based upon CSF α-MSH results in healthy women and a conservative 20% α-MSH change estimate. In various embodiments, a subset of subjects suffered from painful RLS, which accounts for approximately 20% of RLS. This subclassification produced arguably the most interesting finding that CSF β-EDP was lowest in painful RLS, intermediate in nonpainful RLS, and highest in controls. Whereas CSF β-EDP levels differed among the groups, this was not true for plasma β-EDP.

The disclosure includes a method of treating or ameliorating restless leg syndrome using MC4 (melanocortin-4) antagonists. In various embodiments, a method of treating or ameliorating restless leg syndrome (RLS) in a subject in need thereof is provided. The method of treating or ameliorating RLS includes administering to the subject a therapeutically effective amount of an MC4 antagonist. The method of treating or ameliorating RLS includes administering to the subject a therapeutically effective amount of a pharmaceutical composition comprising an MC4 antagonist and at least one pharmaceutically acceptable carrier or excipient.

In various embodiments, a method of reducing symptoms associated with RLS in a subject in need thereof is provided. The method of reducing symptoms associated with RLS includes administering to the subject a therapeutically effective amount an MC4 antagonist. The method of reducing symptoms associated with RLS includes administering to the subject a therapeutically effective amount of a pharmaceutical composition comprising an MC4 antagonist and at least one pharmaceutically acceptable carrier or excipient. In various embodiments, the RLS is nonpainful RLS. In various embodiments, the RLS is painful RLS.

As used herein, the term “reducing symptoms” includes, without limitation, decreasing the severity or intensity of the symptoms, reducing the frequency of the symptoms, reducing the duration of the symptoms, or any combination thereof. Symptoms of RLS include, but are not limited to, unpleasant sensations in the legs, such as creeping, crawling, pulling, or tingling, or urges to move the legs. Other symptoms of RLS can include whole body restlessness, difficulty falling asleep, sleep deprivation, daytime sleepiness, and disturbed sleep. These sensations or symptoms are or can be persistent in the subject.

In various embodiments, a method of treating or ameliorating augmentation associated with treatment of RLS in a subject in need thereof is provided. The method includes administering to the subject a therapeutically effective amount of a pharmaceutical composition comprising an MC4 antagonist and at least one pharmaceutically acceptable carrier or excipient, and wherein the subject was previously administered a dopamine agonist to treat RLS. Augmentation is a phenomenon observed when patients exhibit worsening RLS after treatment with dopamine agonists, for example when symptoms of RLS worsen when the dopamine agonist dose is increased. Dopamine agonists that can lead to augmentation include, but are not limited to, ropinirole, pramipexole, rotigotine, and the like.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats restless leg syndrome. In various embodiments, the MC4 antagonists cross the blood-brain barrier (BBB) and/or to enter the central nervous system and/or the pharmaceutical compositions described herein are formulated to permit the MC4 antagonist to cross the blood-brain barrier (BBB) and/or to enter the central nervous system (CNS).

In certain embodiments, the compositions described herein are administered using diurnal dosing, for example by administering the compositions between 6 PM and 1 AM, or between 6 PM and 10 PM, in a single dose, or by administering a controlled release formulation of the MC4 antagonist such that the peak plasma concentration coincides with the peak MSH concentration and/or peak MC (melanocortin) tone. Peak levels can be determined by, for example, a sleep study where samples from a subject are taken at intervals to measure MSH levels.

In various embodiments, the method described herein includes, prior to treatment, a step of identifying a subject with an elevated ratio of the sum of α-MSH and β-MSH to β-endorphin, and these subjects are subsequently treated according to the methods described herein. For example, in various embodiments, the subject has a (α-MSH+β-MSH):β-endorphin ratio of at least, equal to, or greater than 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, or 17.

In various embodiments, the method described herein includes, prior to treatment, a step of identifying a subject with an elevated ratio of β-MSH to β-endorphin, and these subjects are subsequently treated according to the methods described herein. For example, in various embodiments, the subject has a β-MSH:β-endorphin ratio of at least, equal to, or greater than 3, 3.5, 4, 4.5, 5.5, 6, 6.5, 7, 7.5, 8, 8.5, 9, 9.5, 10, 10.5, or 11.

In certain embodiments, administering the compound(s) described herein to the subject allows for administering a lower dose of the additional therapeutic agent as compared to the dose of the additional therapeutic agent alone that is required to achieve similar results in treating a restless leg syndrome in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating restless leg syndrome. These additional therapeutic agents may comprise compounds that are commercially available or synthetically accessible to those skilled in the art. These additional therapeutic agents are known to treat or reduce the symptoms, of restless leg syndrome.

In certain embodiments, the compounds described herein can be used in combination with radiation therapy. In other embodiments, the combination of administration of the compounds described herein and application of radiation therapy is more effective in treating or preventing restless leg syndrome than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of restless leg syndrome. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat restless leg syndrome in the patient. An effective amount of the therapeutic compound necessary to achieve a therapeutic effect may vary according to factors such as the state of the disease or disorder in the patient; the age, sex, and weight of the patient; and the ability of the therapeutic compound to treat restless leg syndrome in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of restless leg syndrome in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Subjects and Methods

### Study Cohort

Adults at least 18 years of age with moderate-to-severe RLS occurring at least twice weekly were eligible. RLS diagnosis was confirmed by the principal investigator, a board-certified sleep neurologist, using International RLS Study group diagnostic criteria for RLS. Individuals with ambiguous leg symptoms or symptoms suggesting an RLS mimic were excluded. Only persons with primary RLS were included, meaning persons with comorbid Parkinson disease, multiple sclerosis, renal failure, or symptomatic peripheral neuropathy were excluded. Enrolled individuals could not be taking the following medications within 7 days of sample collection: (1) dopaminergic medications, (2) opioids, (3) steroids, (4) amphetamines, or (5) alpha-2-6 ligands. Recreational drugs including opioids (heroin, morphine, methadone), cocaine, and methamphetamine were also exclusionary.

Persons with RLS were further divided into those with painful RLS and those with nonpainful RLS based upon their response to the following question: “Are your RLS symptoms painful? Do the feelings that you experience as RLS hurt, rather than just being uncomfortable?”

Controls were age- and sex-matched to those in the RLS cohort and must have answered “No” to the first two questions of the Cambridge-Hopkins RLS Questionnaire: (1) “Do you have, or have you had, recurrent uncomfortable feelings or sensations in your legs while you are sitting or lying down?” and (2) “Do you have, or have you had, a recurrent need or urge to move your legs while you were sitting or lying down?” RLS was also excluded by personal interview with a board certified neurologist and sleep provider. There were 42 RLS participants and 44 controls.

### Study Visit and Biospecimen Collection

Enrolled subjects arrived at the clinical research unit between 18:00 and 18:30. Urine toxicology was performed to confirm that subjects were not using amphetamines, cocaine, morphine, heroin, or methadone. An intravenous catheter was placed in an antecubital vein by a trained research nurse. Blood (8 mL) was collected in a purple top tube containing ethylenediaminetetraacetic acid (EDTA) at 3 different time points: 19:00, 20:30, and 22:00. To prevent further peptide breakdown, 0.6 trypsin inhibitor units (TIU) aprotinin per milliliter of blood was added. Samples were mixed and placed on ice, then promptly centrifuged at 500×g and 4° C. for 10 minutes. Plasma was then separated, aliquoted, and stored at −70° C. for future peptide analysis. At 21:30, a lumbar puncture was performed using a 22-gauge Whitacre needle with the subject seated upright with the spine flexed. The first 0.5 mL of CSF was discarded. Fourteen milliliters of CSF was collected by gravity in 2 purple top polypropylene EDTA tubes, each containing 5.0 TIU aprotinin. Tubes were mixed by vortex, placed on ice, and promptly centrifuged at 3,000×g and 4° C. for 5 minutes. CSF supernatant was separated, aliquoted, and stored at −70° C. for future peptide analysis.

Following the first blood collection at 19:00, the first suggested immobilization test (SIT) was performed. SIT was performed to assess the propensity to RLS symptom onset and was also used to provoke RLS symptoms; the SIT test was conducted as previously described. Subjects (both RLS and controls) were instructed to sit on a hospital bed with knees flexed at 60-70°. The head of the bed was elevated for comfort. Windows were covered with opaque shading material to account for seasonal variability of outside light. Overhead lights were turned off, and portable ambient lighting was used to light the room at approximately 20-50 lux. Patients were instructed to maintain wakefulness and not to move, save for slight adjustments to avoid discomfort or slight movements of the feet.

Large movements of the body or legs were discouraged. Discomfort from RLS was rated on a Likert scale from 0 to 10, 10 being the most severe, at 10 minutes and every 10 minutes thereafter until the final 60-minute time point. Following the first SIT test, the subject was given a 10-minute break, and then the second SIT test was performed in an identical fashion. RLS symptom severity during each SIT was expressed as the mean of the 6 reported RLS symptom severity ratings. Lumbar puncture was then performed at 21:30 and the final blood draw at 22:00. Finally, pain sensitivity was measured by digital algometry (FPIX; Wagner, Greenwich, CT). The algometer was affixed with a 1 cm² rubber tip and measured pressure applied from this tip to a skin area. Pressure was delivered with increasing levels from 0 to 10 lbs at rv 1 lb/s to the anterior forearm, dorsal hand, and shin, until the subject reported feeling pain. This was repeated 3 times per site, and pressure readings were averaged for each site and then averaged overall to give a total algometer score from 0 to 10 lbs. The assessment of different sites with 3 repetitions at a constant rate of force has been previously described.

### Immunoassays

Plasma was assessed for POMC, ACTH, α-MSH, and β-EDP, and CSF was assessed for POMC, ACTH, α-MSH, β-MSH, and β-EDP. POMC was measured using a custom 2-site enzyme-linked immunosorbent assay (ELISA) with the capture antibody directed to ACTH₁₀₋₁₈ and the detection antibody directed to γ-MSH research resource identifier (RRID: AB_2756529 and AB_2756530). Antibodies have no cross-reactivity with ACTH, α-MSH, β-MSH, or β-EDP. Affinity-purified human 31 K POMC peptide with a sensitivity of 8 fmol/mL was used as a standard. α-MSH was measured using a commercially available ELISA kit for human α-MSH (EKX-KWLBSG; Nordic Biosite, Taby, Sweden), with a sensitivity of 7.5 pg/mL and range of 12.5 to 800 pg/mL with an intra-assay precision coefficient of variation (CV)<8%. β-MSH was measured by radioimmunoassay, using human β-MSH antiserum and isolated human β-MSH peptide for tracer iodination and standards (Phoenix Pharmaceuticals, Burlin-game, CA); there is 10% cross-reactivity with POMC. β-EDP was measured using an ELISA kit for human β-EDP (EKH1392, Nordic Biosite), with a sensitivity of 9.375 pg/mL and range of 15.625 to 1,000 pg/mL with an intra-assay precision CV<8%. ACTH was assayed by double-antibody radio-immunoassay using ¹²⁵I (hACTH Double Antibody RIA kit, 07-106,102; MP Biomedicals, Solon, OH) as per the manufacturer's protocol. All assays were performed in duplicate, and there was >90% agreement between measurements.

### Questionnaires

As an assessment of sleep quality, the Insomnia Severity Index (ISI) was administered to all participants; the ISI is a 7-question scale validated to assess sleep difficulty was scored from 0 to 28. ISI questions assess, in the past 2 weeks, difficulty falling asleep, difficulty staying asleep, sleep satisfaction, and disruption of life quality from sleep difficulty, scored from 0 to 4 as “non,” “mild,” “moderate,” “severe,” and “very severe,” respectively; 0-7=no clinically significant insomnia, 8-14=subthreshold insomnia, 15-21=moderate clinical insomnia, and 22-28=severe clinical insomnia. The Barratt Impulsiveness Scale-11 (BIS) was administered to all participants; the BIS is a 30-item scale validated to assess the level of impulsiveness and impulsive personality traits. Items included statements like “I plan tasks carefully” or “I do things without thinking.” Answer choices were “rarely,” “occasionally,” “often,” and “almost always.” Choices were scored from 1 to 4, with greater impulsiveness indicated by higher scores (range=30-120).

For participants with RLS, RLS severity was assessed using the International RLS Study Group Severity Rating Scale (IRLS), which assesses severity of RLS-related symptoms over the past 2 weeks. The IRLS contains 10 questions that are answered with a score between 0 and 4 for none, mild, moderate, severe, or very severe, respectively. Representative questions include: “Overall, how would you rate the RLS discomfort in your legs or arms?”; “Overall, how severe is your sleep disturbance from your RLS symptoms?”; and “How often do you get RLS symptoms?” (answered as 0=never, 1=1 day per week or less, 2=2-3 days per week, 3=4-5 days per week, 4=6-7 days per week). IRLS scores 1-10, 11-20, 21-30, and 31-40 are considered as mild, moderate, severe, and very severe RLS, respectively.

### Statistical Analysis

Descriptive statistics were conducted to describe categorical variables as number and percentage and continuous variables as mean and standard deviation. Characteristics were compared between the RLS and control groups with χ² tests for categorical variables and Student t tests for comparison of continuous variables between RLS and control groups and analysis of variance for comparison among painful RLS, nonpainful RLS, and control groups; statistical significance was considered as a 2-sided p value<0.05. If comparison among the groups with ANOVA indicated a statistically different result (p<0.05), then post hoc testing was carried out with Student t tests between the different groups. Although there were no data from CSF α-MSH in RLS patients to perform a power analysis, one study of 14 healthy adult women showed a mean±standard deviation CSF α-MSH of 25.5±2.5 pg/mL. Estimating conservatively that a 20% increase in CSF α-MSH in RLS patients with a standard deviation of 7.5 pg/mL would be found, 35 subjects would be needed in each group to find a statistically significant difference. With 36 subjects in each group, there would be 81.2% power to detect a 5.1 pg/mL difference with a standard deviation of 7.5 pg/mL and a significance level of 0.05. Statistical analyses were performed with the R statistical package 4.1.3 (Auckland, New Zealand).

### Characteristics of RLS and Control Groups

A total of 86 individuals participated in the study, 51 being female (42 RLS and 44 control participants). Subjects were middle-aged, with an average age of 51.5±12.8 years. RLS and control cohorts were comparable in age, sex distribution, and body mass index (BMI; Table 1) as were controls, those with painful RLS, and those with nonpainful RLS. Those with RLS scored significantly higher on the ISI, falling in the moderate clinically significant insomnia range compared to controls, who generally did not have clinically significant insomnia. Pain sensitivity as measured by algometry and impulsiveness as measured by the BIS were similar among the groups. On average, participants with RLS scored in the severe range on the IRLS, with an average score of 24.8±9.0; IRLS was similar between those with painful RLS and nonpainful RLS. Nine of the RLS subjects or 21.4% described their RLS symptoms as painful. Those with painful RLS did not differ from those with non-painful RLS or controls in terms of age, sex distribution, or BMI. Pain sensitivity as measured by algometry did not differ in the RLS group compared to controls (8.7±3.0 vs 8.5±2.3, p=0.84), nor did it differ between those with painful RLS and nonpainful RLS (7.8±1.7 vs 8.9±3.3, p=0.26).

TABLE 1
Patient Demographics and Characteristics

| | | Nonpainful | Painful | |
|---|---|---|---|---|
| | Controls, | RLS, | RLS, | |
| Characteristic | n = 44 | n = 33 | n = 9 | p^{a} |
| Age, | 50.4 ± 13.5 | 53.8 ± 12.3 | 50.8 ± 11.2 | 0.52^{b} |
| mean ± SD | | | | |
| BMI, | 26.8 ± 5.0 | 25.7 ± 3.6 | 28.7 ± 5.1 | 0.20^{b} |
| mean ± SD | | | | |
| Female, n (%) | 25 (56.8) | 20 (60.1) | 6 (66.7) | 0.84^{c} |
| ISI | 5.3 ± 6.4 | 14.4 ± 6.7 | 16.8 ± 5.8 | <0.0001^{b} |
| Algometer, lbs | 8.5 ± 2.1 | 8.9 ± 3.3 | 7.8 ± 1.7 | 0.63^{b} |
| Barratt scale | 66.0 ± 10.2 | 67.7 ± 8.1 | 69.9 ± 6.5 | 0.50^{b} |
| IRLS | — | 24.3 ± 9.5 | 26.3 ± 7.3 | 0.50^{d} |

BMI = body mass index in kg/m²; IRLS = International RLS Study Group Severity Rating Scale; ISI = Insomnia Severity Index; RLS = restless legs syndrome.
^{a}Comparing RLS patients to controls; superscript letter indicates type of test used.
^{b}Analysis of variance.
^{c}Chi-squared test.
^{d}Student t test.

### Plasma Hormone Measurements

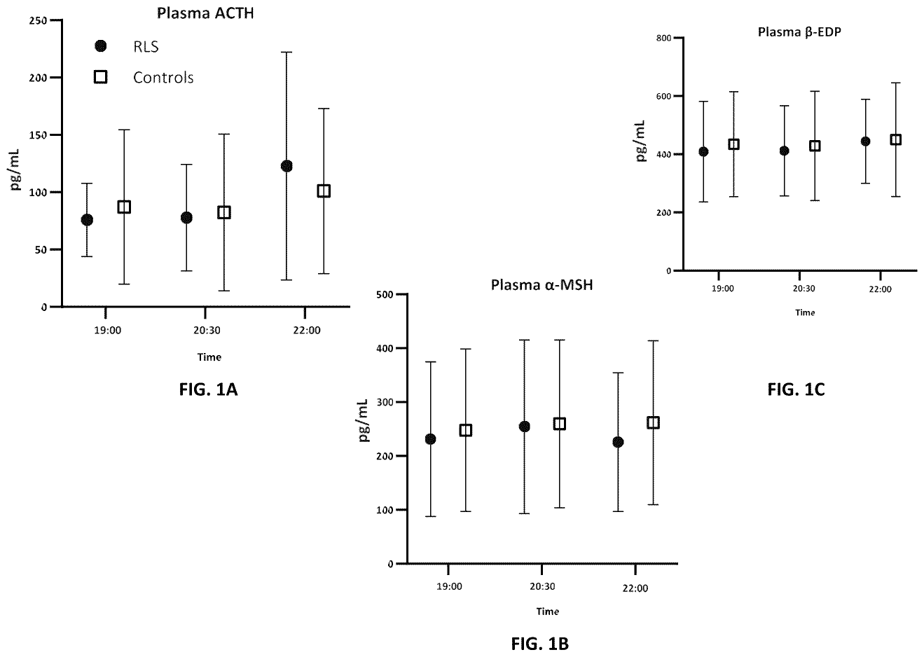

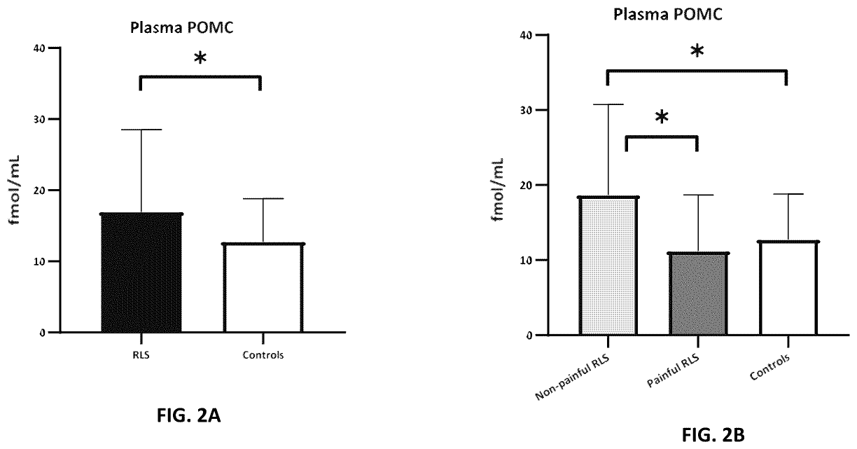

Table 2 shows the average plasma levels of ACTH, α-MSH, and β-EDP for 3 different times points of 19:00, 20:30, and 22:00 and for plasma POMC at 22:00. There was no difference in levels of ACTH, α-MSH, or β-EDP between RLS and control participants at any of the time points. This is also shown graphically in FIGS. 1A-1C. Similarly, there were no differences in levels of ACTH, α-MSH, or β-EDP at any time point among the painful RLS, nonpainful RLS, and control groups (data not shown). Plasma POMC levels in femtomoles per milliliter at 22:00 were significantly greater in RLS (n=39) than in control (n=39) subjects (17.0±11.5 vs 12.7±6.1, p=0.048). Plasma POMC levels were significantly different among those with nonpainful RLS (n=30), those with painful RLS (n=9), and controls (18.7±12.1 vs 11.2±7.4 vs 12.7±6.1, ANOVA p=0.01) with levels being significantly higher in those with non-painful RLS compared to RLS patients with painful RLS (18.7±12.1 vs 11.2±7.4, p=0.04) and also compared to controls (18.7±12.1 vs 12.7±6.1, p=0.02; FIGS. 2A, 2B).

TABLE 2
Plasma Hormone Levels in Controls and RLS Patients

| | Sample Size | | | |
|---|---|---|---|---|
| Plasma Hormone | (controls, RLS) | Controls | RLS | p |
| Plasma ACTH_{19:00}, pg/ml | (40, 42) | 87.2 ± 67.2 | 75.9 ± 32.0 | 0.33 |
| Plasma ACTH_{20:30}, pg/ml | (40, 42) | 82.4 ± 68.2 | 77.8 ± 46.4 | 0.72 |
| Plasma ACTH_{22:00}, pg/ml | (39, 41) | 101.0 ± 71.9 | 122.8 ± 99.4 | 0.27 |
| Plasma α-MSH_{19:00}, pg/ml | (40, 41) | 259.6 ± 155.5 | 254.2 ± 161.0 | 0.62 |
| Plasma α-MSH_{20:30}, pg/ml | (38, 40) | 261.9 ± 151.9 | 231.4 ± 143.5 | 0.88 |
| Plasma α-MSH_{22:00}, pg/ml | (38, 39) | 262.9 ± 151.9 | 225.7 ± 128.7 | 0.26 |
| Plasma β-EDP_{19:00}, pg/ml | (38, 43) | 434.6 ± 180.5 | 409.0 ± 172.7 | 0.52 |
| Plasma β-EDP_{20:30}, pg/ml | (41, 44) | 428.7 ± 187.5 | 412.1 ± 154.8 | 0.66 |
| Plasma β-EDP_{22:00}, pg/ml | (40, 43) | 450.5 ± 195.2 | 444.4 ± 144.1 | 0.87 |
| Plasma POMC_{22:00}, fmol/ml | (39) | 12.7 ± 6.1 | 17.0 ± 11.5 | 0.048^{a} |

ACTH = adrenocorticotropin hormone; POMC = pro-opiomelanocortin; RLS = restless legs syndrome; α-MSH = α-melanocyte-stimulating hor- mone; β-EDP = β-endorphin.
^{a}p < 0.05.

### CSF Hormone Measurements

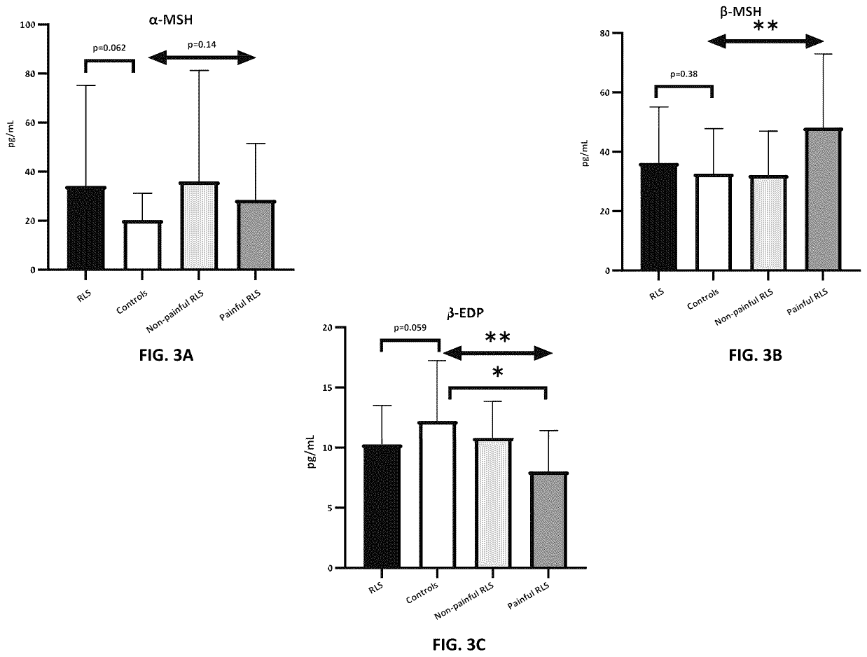

Lumbar puncture was performed at 21:30, and CSF was collected for 37 RLS and 36 control participants. CSF was analyzed for ACTH, α-MSH, β-MSH, β-EDP, and POMC (Table 3). Levels of CSF ACTH (pg/mi) were similar between those with RLS and controls (22.0±10.6 vs 24.4±14.0, p=0.42) and among controls, painful RLS sufferers, and nonpainful RLS sufferers. CSF α-MSH trended toward being higher in RLS patients compared to controls (34.2±40.9 vs 20.3±11.0, p=0.062). CSF-MSH was highest in nonpainful RLS sufferers, intermediate in those with painful RLS, and lowest in controls, although this was not statistically significant (36.0±45.3 vs 28.5±23.0 vs 20.3±11.0, ANOVA p=0.14; FIGS. 3A-C).

TABLE 3
CSF Hormone Levels in Controls and RLS Patients

| | Sample Size | | | | | | |
|---|---|---|---|---|---|---|---|
| | (controls, RLS, | | | | | | |
| | nonpainful RLS, | Controls, | All RLS, | | Nonpainful | Painful | |
| CSF Analyte | painful RLS) | n = 36 | n = 37 | p^{a} | RLS | RLS | p^{b} |
| ACTH | (35, 37, 28, 9) | 24.4 ± 14.0 | 22.0 ± 10.6 | 0.42 | 21.4 ± 11.1 | 24.0 ± 9.1 | 0.62 |
| α-MSH | (34, 34, 26, 8) | 20.3 ± 10.9 | 34.2 ± 40.9 | 0.062 | 36.0 ± 45.3 | 28.5 ± 23.0 | 0.14 |
| β-MSH | (35, 35, 26, 9) | 32.6 ± 15.2 | 36.2 ± 18.9 | 0.38 | 32.1 ± 14.8 | 48.2 ± 24.8 | 0.03^{c} |
| β-EDP | (34, 35, 27, 7) | 12.3 ± 5.0 | 10.3 ± 3.2 | 0.059 | 10.8 ± 3.1 | 8.0 ± 3.4 | 0.049^{c} |
| POMC | (36, 37, 28, 9) | 241.9 ± 108 | 225.5 ± 69 | 0.44 | 228.5 ± 68 | 216 ± 74.0 | 0.70 |
| α-MSH:β-EDP | (33, 32, 25, 7) | 1.90 ± 1.72 | 3.45 ± 3.8 | 0.03^{c} | 3.37 ± 3.93 | 3.73 ± 3.53 | 0.09 |
| β-MSH:β-EDP | (33, 32, 25, 7) | 3.18 ± 2.08 | 4.00 ± 2.71 | 0.19 | 3.18 ± 1.77 | 6.87 ± 3.63 | 0.0004^{d} |
| (α-MSH + β- | (32, 30, 23, 7) | 5.15 ± 2.95 | 7.66 ± 5.14 | 0.02^{c} | 6.77 ± 4.56 | 10.6 ± 6.18 | 0.007^{e} |
| MSH):β-EDP | | | | | | | |

“:” indicates ratio.
ACTH = adrenocorticotropin hormone; CSF = cerebrospinal fluid; POMC = pro-opiomelanocortin; RLS = restless legs syndrome; α-MSH = α- melanocyte-stimulating hormone; β-EDP = β-endorphin.
^{a}Student t test comparing means for all RLS patients to controls.
^{b}Analysis of variance comparing means among nonpainful RLS, painful RLS, and controls.
^{c}p < 0.05.
^{d}p < 0.001.
^{e}p < 0.01.

CSF β-MSH (pg/mL) was similar between RLS subjects and controls (36.2±18.9 vs 32.6±15.2, p=0.38), but when RLS subjects were subclassified, CSF β-MSH was higher in those with painful RLS than in nonpainful RLS or in controls (48.2±24.8 vs 32.1±14.8 vs 32.6±15.2, ANOVA p=0.03; FIG. 3). CSF β-EDP (pg/mL) trended toward being lower in the RLS compared to the control cohort (10.3±3.2 vs 12.3±5.0, p=0.059), and was significantly different among the different cohorts, with those with painful RLS having the lowest, nonpainful RLS having intermediate, and controls having the highest CSF β-EDP (8.0±3.4 vs 10.8±3.1 vs 12.3±5.0, ANOVA p=0.049; FIG. 3). With post hoc testing, those with painful RLS had significantly lower CSF β-EDP than controls (8.0±3.4 vs 12.3±5.0, p=0.02). In the overall cohort, severity of RLS as determined by the IRLS did not correlate significantly to levels of any hormone. In women with RLS, severity of RLS by IRLS correlated positively with CSF β-MSH (r=+0.57, p=0.007) and marginally with CSF ACTH (r=+0.40, p=0.059), but not levels of other CSF hormones. Also in women, CSF β-MSH correlated with severity of RLS symptoms in the first SIT (r=+0.48, p=0.04) but not the second SIT (r=+0.31, p=0.19). In men with RLS, RLS severity by IRLS correlated negatively with CSF POMC (r=−0.55, p=0.0501) but not other CSF hormones.

Ratios of the MSH hormones to β-EDP were calculated and compared among the groups. The ratio of α-MSH to β-EDP was significantly higher for those with RLS than for controls (3.45±3.8 vs 1.90±1.72, p=0.03). The ratio of β-MSH to β-EDP was similar in RLS and control cohorts, but was significantly higher in those with painful RLS than those with nonpainful RLS or controls (6.87±3.63 vs 3.18±1.77 vs 3.18±2.08, ANOVA p=0.0004). The ratio of the sum of α-MSH and β-MSH to β-EDP was significantly higher in those with RLS compared to controls (7.66±5.14 vs 5.15±2.95, p=0.02) and was highest in painful RLS, intermediate in nonpainful RLS, and lowest in controls (10.6±6.18 vs 6.77±4.56 vs 5.15±2.95, p=0.007).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### ENUMERATED EMBODIMENTS

The following enumerated embodiments are provided, the numbering of which is not to be construed as designating levels of importance:

Embodiment 1 provides a method of treating, preventing, and/or ameliorating restless leg syndrome (RLS) in a subject in need thereof, the method comprising administering to the subject a therapeutically effective amount of an MC4 antagonist or a pharmaceutically acceptable salt thereof, which is optionally formulated as a pharmaceutical composition further comprising at least one pharmaceutically acceptable carrier or excipient.

Embodiment 2 provides the method of embodiment 1, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 3 provides the method of any one of embodiments 1-2, further comprising administering at least one additional therapeutic agent to the subject.

Embodiment 4 provides the method of any one of embodiments 1-3, wherein the at least one additional therapeutic agent is administered to the subject sequentially or concurrently with the compound.

Embodiment 5 provides the method of any one of embodiments 1-4, wherein the subject is human.

Embodiment 6 provides the method of any one of embodiments 1-5, wherein the MC4 antagonist is a small-molecule, peptide, or antibody.

Embodiment 7 provides a method of reducing and/or eliminating symptoms associated with RLS in a subject in need thereof, the method comprising administering to the subject a therapeutically effective amount of an MC4 antagonist or a pharmaceutically acceptable salt thereof, which is optionally formulated as a pharmaceutical composition further comprising at least one pharmaceutically acceptable carrier or excipient.

Embodiment 8 provides the method of embodiment 7, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 9 provides the method of any one of embodiments 7-8, further comprising administering to the subject at least one additional therapeutic agent.

Embodiment 10 provides the method of any one of embodiments 7-9, wherein the at least one additional therapeutic agent is administered to the subject sequentially or concurrently with the compound.

Embodiment 11 provides the method of any one of embodiments 7-10, wherein the subject is human.

Embodiment 12 provides the method of any one of embodiments 7-11, wherein the MC4 antagonist is a small-molecule, peptide, or antibody.

Embodiment 13 provides a method of treating, preventing, and/or ameliorating augmentation associated with prior treatment of RLS in a subject in need thereof, the method comprising administering to the subject a therapeutically effective amount of an MC4 antagonist or a pharmaceutically acceptable salt thereof, which is optionally formulated as a pharmaceutical composition further comprising at least one pharmaceutically acceptable carrier or excipient; wherein the prior treatment is a previously administered dopamine agonist to the subject.

Embodiment 14 provides the method of embodiment 1, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 15 provides the method of any one of embodiments 13-14, further comprising administering to the subject at least one additional therapeutic agent.

Embodiment 16 provides the method of any one of embodiments 13-15, wherein the at least one additional therapeutic agent is administered to the subject sequentially or concurrently with the compound.

Embodiment 17 provides the method of any one of embodiments 13-16, wherein the subject is human.

Embodiment 18 provides the method of any one of embodiments 13-17, wherein the MC4 antagonist is a small-molecule, peptide, or antibody.

Embodiment 19 provides the method of any one of embodiments 1-18, which further comprises identifying a subject with a (α-MSH+β-MSH):β-endorphin ratio of at least about 5 prior to administering the MC4 antagonist to the subject.

Embodiment 20 provides the method of any one of embodiments 1-18, which further comprises identifying a subject with a β-MSH:β-endorphin ratio of at least about 3 prior to administering the MC4 antagonist to the subject.

What is claimed is:

1. A method of treating, preventing, or ameliorating restless leg syndrome (RLS) in a subject in need thereof, the method comprising:
administering to the subject a therapeutically effective amount of an MC4 antagonist or a pharmaceutically acceptable salt thereof, which is optionally formulated as a pharmaceutical composition further comprising at least one pharmaceutically acceptable carrier or excipient.

2. The method of claim 1, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

3. The method of claim 1, further comprising administering at least one additional therapeutic agent to the subject.

4. The method of claim 3, wherein the at least one additional therapeutic agent is administered to the subject sequentially or concurrently with the compound.

5. The method of claim 1, wherein the subject is human.

6. The method of claim 1, wherein the MC4 antagonist is a small-molecule, peptide, or antibody.

7. A method of reducing or eliminating symptoms associated with RLS in a subject in need thereof, the method comprising:
administering to the subject a therapeutically effective amount of an MC4 antagonist or a pharmaceutically acceptable salt thereof, which is optionally formulated as a pharmaceutical composition further comprising at least one pharmaceutically acceptable carrier or excipient.

8. The method of claim 7, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

9. The method of claim 7, further comprising administering to the subject at least one additional therapeutic agent.

10. The method of claim 9, wherein the at least one additional therapeutic agent is administered to the subject sequentially or concurrently with the compound.

11. The method of claim 7, wherein the subject is human.

12. The method of claim 7, wherein the MC4 antagonist is a small-molecule, peptide, or antibody.

13. A method of treating, preventing, or ameliorating augmentation associated with prior treatment of RLS in a subject in need thereof, the method comprising:
administering to the subject a therapeutically effective amount of an MC4 antagonist or a pharmaceutically acceptable salt thereof, which is optionally formulated as a pharmaceutical composition further comprising at least one pharmaceutically acceptable carrier or excipient;
wherein the prior treatment is a previously administered dopamine agonist to the subject.

14. The method of claim 13, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

15. The method of claim 13, further comprising administering to the subject at least one additional therapeutic agent.

16. The method of claim 15, wherein the at least one additional therapeutic agent is administered to the subject sequentially or concurrently with the compound.

17. The method of claim 13, wherein the subject is human.

18. The method of claim 13, wherein the MC4 antagonist is a small-molecule, peptide, or antibody.

19. The method of claim 1, which further comprises identifying a subject with a (α-MSH+β-MSH):β-endorphin ratio of at least about 5 prior to administering the MC4 antagonist to the subject.

20. The method of claim 1, which further comprises identifying a subject with a β-MSH:β-endorphin ratio of at least about 3 prior to administering the MC4 antagonist to the subject.


SEQUENCE LISTING (2 sequences)

SEQ ID NO: 1 - protein, 9 aa, synthetic construct
  SITE 2: note = Nle
  SITE 5: note = D-Naphthylalanine
  SITE 1: note = Acetylation of the N-terminus.
CXRHXRWGC

SEQ ID NO: 2 - protein, 9 aa, synthetic construct
  SITE 1: note = Nle
  SITE 4: note = D-Naphthylalanine
  REGION 2..7: note = This is a cyclic region.
  SITE 8: note = D-amino acid
  SITE 9: note = D-amino acid
  SITE 1: note = Acetylation of the N-terminus.
XDPXRWKVP